(19) United States
(12) Patent Application Publication
(10) Pub. No.: US 2013/0053565 A1
(43) Pub. Date: Feb. 28, 2013

(54) SUBSTITUTED 3-PIPERIDONE COMPOUNDS

(71) Applicants: Janis Louie, Salt Lake City, UT (US); Puneet Kumar, Salt Lake City, UT (US)

(72) Inventors: Janis Louie, Salt Lake City, UT (US); Puneet Kumar, Salt Lake City, UT (US)

(73) Assignee: UNIVERSITY OF UTAH RESEARCH FOUNDATION, Salt Lake City, UT (US)

(21) Appl. No.: 13/598,116

(22) Filed: Aug. 29, 2012

Related U.S. Application Data
(60) Provisional application No. 61/528,738, filed on Aug. 29, 2011.
(60) Provisional application No. 61/615,517, filed on Mar. 26, 2012.

(51) Int. Cl. C07D 211/86; C07F 7/10; C07F 7/22; C07D 211/96

(52) U.S. Cl. 546/4; 546/249; 546/14; 546/298; 546/290

(57) ABSTRACT

Described herein are methods for synthesizing substituted 3-piperidone compounds. Notably, substituted 3-piperidones can also be prepared in enantiopure form. The methods may allow for preparation of highly substituted piperidine cores. Also disclosed are 3-piperidone compounds and pharmaceutical compositions comprising the compounds.

### CROSS-REFERENCE TO RELATED APPLICATIONS

This application claims priority to U.S. Provisional Patent Application No. 61/528,738, filed on Aug. 29, 2011, and U.S. Provisional Patent Application No. 61/615,517, filed on Mar. 26, 2012, the entire contents of each which are hereby incorporated by reference.

### STATEMENT REGARDING FEDERALLY SPONSORED RESEARCH

This invention was made with U.S. Government support awarded by the National Science Foundation, Grant No. 0911017. The U.S. Government has certain rights in this invention.

### BACKGROUND

The ubiquity of piperidines in pharmaceuticals and natural products makes them attractive targets for organic synthesis. While progress has been made in accessing substituted piperidines, the synthesis of highly substituted piperidines is still a challenging problem. Also, most of the existing strategies rely on multi-step routes. Furthermore, while synthetic methods for accessing 2- and 4-piperidones are well-established, highly efficient methods for preparing 3-piperidones are lacking. There is a need for operationally simple, expeditious, and efficient methodologies to access these heterocycles.

### SUMMARY

In one aspect, this disclosure provides a method of synthesizing a compound of formula (I):

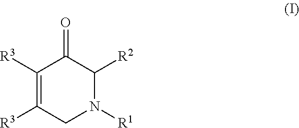

wherein:

R¹ is selected from the group consisting of hydrogen, alkyl or a nitrogen protecting group;

R² is selected from the group consisting of hydrogen, alkyl, alkenyl, alkynyl, cycloalkyl, heterocyclyl, aryl, heteroaryl, silyl and stannyl, any of which may be optionally substituted;

each R³ is independently selected from the group consisting of hydrogen, alkyl, alkenyl, alkynyl, cycloalkyl, heterocyclyl, aryl, heteroaryl, silyl and stannyl, any of which may be optionally substituted, or both R³ are taken together with the atoms to which they are attached to form an optionally substituted ring;

the method comprising combining the following components to form a reaction mixture:

a) a compound of formula (II):

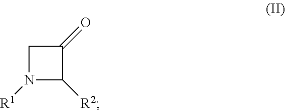

b) a compound of formula (III):

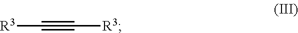

c) a nickel-containing compound; and

d) a ligand.

In another aspect, this disclosure provides a compound of formula (I):

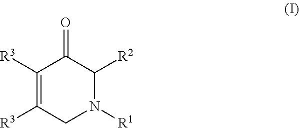

wherein:

R¹ is selected from the group consisting of hydrogen, alkyl or a nitrogen protecting group; and

R² is selected from the group consisting of hydrogen, alkyl, alkenyl, alkynyl, cycloalkyl, heterocyclyl, aryl, heteroaryl, silyl and stannyl, any of which may be optionally substituted; and

each R³ is independently selected from the group consisting of hydrogen, alkyl, alkenyl, alkynyl, cycloalkyl, heterocyclyl, aryl, heteroaryl, silyl and stannyl, any of which may be optionally substituted, or both R³ are taken together with the atoms to which they are attached to form an optionally substituted ring.

Other aspects and embodiments will become apparent in light of the following description and drawings.

### BRIEF DESCRIPTION OF THE DRAWINGS

FIG. 1 shows an Oak Ridge Thermal Ellipsoid Plot (ORTEP) of compound 2c, described herein, which was characterized by single crystal X-ray crystallography.

### DETAILED DESCRIPTION

Described herein are methods of synthesizing substituted 3-piperidone compounds, which may be subsequently converted to substituted piperidines. 3-piperidone compounds can be effectively produced in one step, by coupling a 3-aza-cyclobutanone compound with an alkyne in the presence of a nickel-containing compound and a ligand. These cycloaddition reactions proceed with reasonably low catalyst loading and ligand loading, at moderate temperatures and require only several hours. The methodology may be of great use in developing new pharmaceutical compounds, as existing methods of preparing highly substituted piperidine compounds as well as 3-piperidone core structures are limited.

### 1. DEFINITIONS

The terminology used herein is for the purpose of describing particular embodiments only and is not intended to be limiting. As used in the specification and the appended claims, the singular forms “a,” “and” and “the” include plural references unless the context clearly dictates otherwise.

Section headings as used in this section and the entire disclosure herein are not intended to be limiting.

For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

As used herein, the term “about” is used synonymously with the term “approximately.” Illustratively, the term “about,” as used in connection with a particular value, indicates that the value may be slightly outside the particular value. Variation may be due to conditions such as experimental error, manufacturing tolerances, variations in equilibrium conditions, and the like. In some embodiments, the term “about” includes the cited value plus or minus 10%. Such values are thus encompassed by the scope of the claims reciting the terms “about” and “approximately.”

Definitions of specific functional groups and chemical terms are described in more detail below. For purposes of this disclosure, the chemical elements are identified in accordance with the Periodic Table of the Elements, CAS version, Handbook of Chemistry and Physics, 75th Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “acyl” refers to an alkylcarbonyl, cycloalkylcarbonyl, heterocyclylcarbonyl, arylcarbonyl or heteroarylcarbonyl substituent, any of which may be further substituted (e.g., with one or more substituents).

The term “alkyl” refers to a straight or branched saturated hydrocarbon chain. Alkyl groups may include a specified number of carbon atoms. For example, C₁-C₁₂ alkyl indicates that the alkyl group may have 1, 2, 3, 4, 5, 6, 7, 8, 9, 10, 11 or 12 carbon atoms. An alkyl group may be, e.g., a C₁-C₁₂ alkyl group, a C₁-C₁₀ alkyl group, a C₁-C₈ alkyl group, a C₁-C₆ alkyl group or a C₁-C₄ alkyl group. For example, exemplary C₁-C₄ alkyl groups include methyl, ethyl, n-propyl, isopropyl, n-butyl, sec-butyl, isobutyl and tert-butyl groups. An alkyl group may be optionally substituted with one or more substituents.

The term “alkenyl” refers to a straight or branched hydrocarbon chain having one or more double bonds. Alkenyl groups may include a specified number of carbon atoms. For example, C₂-C₁₂ alkenyl indicates that the alkenyl group may have 2, 3, 4, 5, 6, 7, 8, 9, 10, 11 or 12 carbon atoms. An alkenyl group may be, e.g., a C₂-C₁₂ alkenyl group, a C₂-C₁₀ alkenyl group, a C₂-C₈ alkenyl group, a C₂-C₆ alkenyl group or a C₂-C₄ alkenyl group. Examples of alkenyl groups include but are not limited to allyl, propenyl, 2-butenyl, 3-hexenyl and 3-octenyl groups. One of the double bond carbons may optionally be the point of attachment of the alkenyl substituent. An alkenyl group may be optionally substituted with one or more substituents.

The term “alkynyl” refers to a straight or branched hydrocarbon chain having one or more triple bonds. Alkynyl groups may include a specified number of carbon atoms. For example, C₂-C₁₂ alkynyl indicates that the alkynyl group may have 2, 3, 4, 5, 6, 7, 8, 9, 10, 11 or 12 carbon atoms. An alkynyl group may be, e.g., a C₂-C₁₂ alkynyl group, a C₂-C₁₀ alkynyl group, a C₂-C₈ alkynyl group, a C₂-C₆ alkynyl group or a C₂-C₄ alkynyl group. Examples of alkynyl groups include but are not limited to ethynyl, propargyl, and 3-hexynyl. One of the triple bond carbons may optionally be the point of attachment of the alkynyl substituent. An alkynyl group may be optionally substituted with one or more substituents.

The term “aryl” refers to an aromatic monocyclic, bicyclic, or tricyclic hydrocarbon ring system, wherein any ring atom capable of substitution may be substituted (e.g., with one or more substituents). Examples of aryl moieties include but are not limited to phenyl, naphthyl, and anthracenyl. Aryl groups may be optionally substituted with one or more substituents.

The term “arylalkyl” refers to an alkyl moiety in which at least one alkyl hydrogen atom is replaced with an aryl group. Arylalkyl includes groups in which more than one hydrogen atom has been replaced with an aryl group. Examples of arylalkyl groups include but are not limited to benzyl, 2-phenylethyl, 3-phenylpropyl, 9-fluorenyl, benzhydryl, and trityl groups. Arylalkyl groups may be optionally substituted with one or more substituents, on either the aryl moiety or the alkyl moiety.

The term “cycloalkyl” as used herein refers to non-aromatic, saturated or partially unsaturated cyclic, bicyclic, tricyclic or polycyclic hydrocarbon groups having 3 to 12 carbons. Any ring atom may be substituted (e.g., with one or more substituents). Cycloalkyl groups may contain fused rings. Fused rings are rings that share one or more common carbon atoms. Examples of cycloalkyl groups include but are not limited to cyclopropyl, cyclobutyl, cyclopentyl, cyclohexyl, cyclohexenyl, cyclohexadienyl, methylcyclohexyl, adamantyl, norbornyl, norbornenyl, tetrahydronaphthalenyl and dihydroindenyl. Cycloalkyl groups may be optionally substituted with one or more substituents.

The term “halo” or “halogen” as used herein refers to any radical of fluorine, chlorine, bromine or iodine.

The term “haloalkyl” as used herein refers to an alkyl group as defined herein, such as a C₁-C₄ alkyl group, in which one or more hydrogen atoms are replaced with halogen atoms, and includes alkyl moieties in which all hydrogens have been replaced with halogens (e.g., perfluoroalkyl such as CF₃).

The term “heteroaryl” as used herein refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms independently selected from O, N, S, P and Si (e.g., carbon atoms and 1-3, 1-6, or 1-9 heteroatoms independently selected from O, N, S, P and Si if monocyclic, bicyclic, or tricyclic, respectively). Any ring atom may be substituted (e.g., with one or more substituents). Heteroaryl groups may contain fused rings, which are rings that share one or more common atoms. Examples of heteroaryl groups include but are not limited to radicals of pyridine, pyrimidine, pyrazine, pyridazine, pyrrole, imidazole, pyrazole, oxazole, isoxazole, furan, thiazole, isothiazole, thiophene, quinoline, isoquinoline, quinoxaline, quinazoline, cinnoline, indole, isoindole, indolizine, indazole, benzimidazole, phthalazine, pteridine, carbazole, carboline, phenanthridine, acridine, phenanthroline, phenazine, naphthyridines and purines. Heteroaryl groups may be optionally substituted with one or more substituents.

The term “heteroatom”, as used herein, refers to a non-carbon or hydrogen atom such as a nitrogen, sulfur, oxygen, silicon or phosphorus atom. Groups containing more than one heteroatom may contain different heteroatoms.

The term “heterocyclyl”, as used herein, refers to a nonaromatic, saturated or partially unsaturated 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, S, Si and P (e.g., carbon atoms and 1-3, 1-6, or 1-9 heteroatoms of O, N, S, Si and P if monocyclic, bicyclic, or tricyclic, respectively). Any ring atom may be substituted (e.g., with one or more substituents). Heterocyclyl groups may contain fused rings, which are rings that share one or more common atoms. Examples of heterocyclyl groups include but are not limited to radicals of tetrahydrofuran, tetrahydrothiophene, tetrahydropyran, oxetane, piperidine, piperazine, morpholine, pyrroline, pyrimidine, pyrrolidine, indoline, tetrahydropyridine, dihydropyran, thianthrene, pyran, benzopyran, xanthene, phenoxathiin, phenothiazine, furazan, lactones, lactams such as azetidinones and pyrrolidinones, sultams, sultones, and the like. Heterocyclyl groups may be optionally substituted with one or more substituents.

The term “hydroxy” refers to an —OH radical. The term “alkoxy” refers to an —O-alkyl radical. The term “aryloxy” refers to an —O-aryl radical. The alkyl portion of an alkoxy group or the aryl portion of an aryloxy group may be optionally substituted with one or more substituents. (For example, “alkoxy” encompasses hydroxyalkoxy groups, in which the alkyl portion of the alkoxy group is substituted with a hydroxy group.)

The term “ligand” refers to an organic molecule comprising at least one unshared electron pair that is available for donation to a metal atom. The unshared electron pair may reside on, for example, a nitrogen, phosphorus, arsenic, oxygen, sulfur or carbon atom.

The term “nitrogen protecting group” refers to a moiety that is used to temporarily block a desired nitrogen functional group in a compound, e.g., a compound with multiple reactive sites. The nitrogen protecting group may protect a nitrogen atom from undesirable chemical reactions under specified conditions (e.g., pH, temperature, radiation, solvent, and the like). In some embodiments, a protecting group has one, or more, or all of the following characteristics: a) it may be added selectively to a nitrogen functional group in good yield to give a protected compound; b) it is stable to reactions occurring at one or more reactive sites in the molecule; and c) it is selectively removable in good yield by reagents that do not attack the regenerated, deprotected nitrogen functional group. Nitrogen protecting groups include but are not limited to: acetyl (Ac), allyloxycarbonyl (Alloc), benzyl (Bn), benzhydryl (Bnh), benzoyl (Bz), tert-butyloxycarbonyl (Boc), 2-biphenyl-2-propoxycarbonyl (Bpoc), carbobenzyloxy (Cbz), 3,4-dimethoxybenzyl (DMPM), 9-fluorenylmethyloxycarbonyl (FMOC), p-methoxybenzylcarbonyl (Moz), 2- or 4-nitrobenzenesulfonyl (nosyl, Ns), o-nitrophenlysulfenyl (Nps), 2-(phenylsulfonyl)ethyloxycarbonyl (Psec), p-ethoxybenzyl (PMB), p-methoxyphenyl (PMP), p-toluenesulfonyl (tosyl, Ts), 6-nitroveratryloxycarbonyl (Nvoc), 2-trimethylsilylethyloxycarbonyl (Teoc) and 2,2,2-trichloroethyloxycarbonyl (Troc), and, in suitable cases (e.g., cyclic amines), a nitroxide radical. Other examples may be found in, for example, Protective Groups in Organic Synthesis (T. Green and P. Wuts; 3rd Edition; John Wiley and Sons, 1999).

The term “oxo” refers to an oxygen atom, which forms a carbonyl when attached to carbon, an N-oxide when attached to nitrogen, and a sulfoxide or sulfone when attached to sulfur (i.e. ═O).

The term “mercapto” or “thiol” refers to an —SH radical. The term “thioalkoxy” or “thioether” refers to an —S-alkyl radical. The term “thioaryloxy” refers to an —S-aryl radical.

The term “substituents” refers to a group “substituted” on an alkyl, alkenyl, alkynyl, cycloalkyl, heterocyclyl, aryl, arylalkyl, heteroaryl or heteroarylalkyl group at any atom of that group. Any atom may be substituted. Suitable substituents include, without limitation: acyl, acylamido, acyloxy, alkoxy, alkyl, alkenyl, alkynyl, amido, amino, carboxy, cyano, ester, halo, haloalkyl, hydroxy, imino, nitro, oxo (e.g., C═O), phosphonate, sulfinyl, sulfonyl, sulfonate, sulfonamino, sulfonamido, thioamido, thiol, thioxo (e.g., C═S), and ureido. In some embodiments, substituents on a group are independently any one single, or any combination of the aforementioned substituents. In some embodiments, a substituent may itself be substituted with any one of the above substituents.

The above substituents may be abbreviated herein. For example, the abbreviations Me, Et, Ph and Bn represent methyl, ethyl, phenyl and benzyl, respectively. A more comprehensive list of standard abbreviations used by organic chemists appears in a table entitled Standard List of Abbreviations of the Journal of Organic Chemistry. The abbreviations contained in said list are hereby incorporated by reference.

For compounds described herein, groups and substituents thereof may be selected in accordance with permitted valence of the atoms and the substituents, and such that the selections and substitutions result in a stable compound, e.g., a compound that does not spontaneously undergo transformation such as by rearrangement, cyclization, elimination, etc.

Where substituent groups are specified by their conventional chemical formulae, written from left to right, they optionally encompass substituents resulting from writing the structure from right to left, e.g., —CH₂O— optionally also recites —OCH₂—.

In accordance with a convention used in the art, the group:

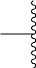

is used in structural formulae herein to depict the bond that is the point of attachment of the moiety or substituent to the core or backbone structure.

### 2. METHODS OF SYNTHESIZING COMPOUNDS

This disclosure provides methods of synthesizing compounds of formula (I), which may proceed via coupling of a 3-aza-cyclobutanone compound with an alkyne. In particular, the disclosure provides a method of synthesizing a compound of formula (I):

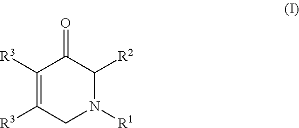

wherein:

R¹ is selected from the group consisting of hydrogen, alkyl or a nitrogen protecting group;

R² is selected from the group consisting of hydrogen, alkyl, alkenyl, alkynyl, cycloalkyl, heterocyclyl, aryl, heteroaryl, silyl and stannyl, any of which may be optionally substituted;

each R³ is independently selected from the group consisting of hydrogen, alkyl, alkenyl, alkynyl, cycloalkyl, heterocyclyl, aryl, heteroaryl, silyl and stannyl, any of which may be optionally substituted, or both R³ are taken together with the atoms to which they are attached to form an optionally substituted ring;

the method comprising combining the following components to form a reaction mixture:

a) a compound of formula (II):

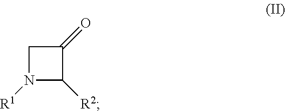

b) a compound of formula (III):

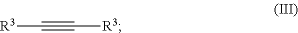

c) a nickel-containing compound; and

d) a ligand.

a. Compounds of Formula (II)

One of the starting materials in the methods of synthesis described herein is a compound of formula (II):

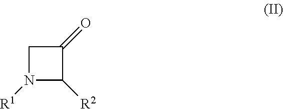

wherein:

R¹ is selected from the group consisting of hydrogen, alkyl or a nitrogen protecting group; and

R² is selected from the group consisting of hydrogen, alkyl, alkenyl, alkynyl, cycloalkyl, heterocyclyl, aryl, heteroaryl, silyl and stannyl, any of which may be optionally substituted.

In some embodiments, R¹ is alkyl. In some embodiments, R¹ is a nitrogen protecting group, such as, e.g., tert-butyloxycarbonyl (Boc) or tosyl (Ts).

In some embodiments, R² is hydrogen. In some embodiments, R² is a substituted alkyl group such as an arylalkyl group.

Suitable compounds of formula (II) include, but are not limited to, 1-Boc-3-azetidinone and 2-benzyl-3-Boc-azetidinone. Such compounds may be commercially available from a variety of sources (e.g., Sigma-Aldrich, St. Louis, Mo.), or may be synthesized by any means known in the art. For example, 3-azetidinone may be N-protected using any suitable protecting group reagent. 2-benzyl-3-Boc-azetidinone may be prepared, for example, from Boc-protected phenylalanine amino acid.

b. Compounds of Formula (III)

Another starting material in the methods of synthesis described herein is a compound of formula (III):

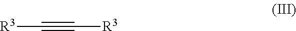

wherein:

each R³ is independently selected from the group consisting of hydrogen, hydrogen, alkyl, alkenyl, alkynyl, cycloalkyl, heterocyclyl, aryl, heteroaryl, silyl and stannyl, any of which may be optionally substituted, or both R³ are taken together with the atoms to which they are attached to form an optionally substituted ring.

In some embodiments, the two R³ groups are the same. In some embodiments, the two R³ groups are different.

In some embodiments, each R³ is independently selected from the group consisting of hydrogen, C₁-C₆ alkyl (e.g., C₁-C₄ alkyl such as methyl, ethyl, isopropyl, n-propyl and tert-butyl), C₂-C₆ alkenyl (e.g., C₂-C₄ alkenyl such as propenyl, such as prop-1-en-2-yl), silyl (e.g., trimethylsilyl), aryl (e.g., phenyl) including substituted aryl groups (e.g., phenyl substituted with alkoxy such as methoxy, or haloalkyl such as trifluoromethyl), heteroaryl (e.g., thienyl or furanyl) including substituted heteroaryl groups, and stannyl (e.g., tributylstannyl).

In some embodiments, both R³ are taken together with the atoms to which they are attached to form an optionally substituted ring. In some embodiments, both R³ are taken together with the atoms to which they are attached to form an unsubstituted ring. The ring may include at least 8 atoms. The ring may include up to 12 atoms.

Suitable compounds of formula (III) include, but are not limited to, 2-butyne, oct-4-yne, 4,4-dimethylpent-2-yne, 3,3-dimethylbut-1-yne, trimethyl(prop-1-ynyl)silane, tributyl(prop-1-ynyl)stannane, 2-methylhex-1-en-3-yne, 1,2-diphenylethyne, prop-1-ynylbenzene, 1-methoxy-4-(prop-1-ynyl)benzene, 1-(prop-1-ynyl)-4-(trifluoromethyl)benzene, trimethyl(phenylethynyl)silane, trimethyl(thiophen-3-ylethynyl)silane, (furan-3-ylethynyl)trimethylsilane, tributyl(phenylethynyl)stannane, cyclooctyne and cyclododecyne. Such compounds may be commercially available from a variety of sources (e.g., Sigma-Aldrich, St. Louis, Mo.), or may be synthesized by any means known in the art.

A compound of formula (III) may be included in a reaction at an amount of about 1.0 to about 5.0 equivalents compared to the compound of formula (II). For example, about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0 equivalents of the compound of formula (III) may be used compared to the compound of formula (II). By way of another example, if 1.0 mmol of a compound of formula (II) is included in a reaction mixture, then about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9 or 5.0 mmol of the compound of formula (III) may be included in the reaction mixture.

c. Nickel-Containing Compounds

Another starting material in the methods of synthesis described herein is a nickel-containing compound. For example, the nickel-containing compound may include Ni(0). A suitable source of Ni(0) is bis(cyclooctadiene)nickel(0).

A nickel-containing compound may be included in a reaction mixture in an amount of about 1 mol % to about 10 mol %, about 1 mol % to less than about 10 mol %, about 1 mol % to less than about 9 mol %, or about 1 mol % to less than about 8 mol %. For example, a nickel-containing compound may be included in a reaction mixture in an amount of about 1, 1.5, 2, 2.5, 3, 3.5, 4, 4.5, 5, 5.5, 6, 6.5, 7, 7.5, 8, 8.5, 9, 9.5 or 10 mol %.

d. Ligand

Another starting material in the methods of synthesis described herein is a ligand. The ligand includes at least one atom bearing an unshared electron pair, which may interact with the nickel of the nickel-containing compound. For example, a ligand may include at least one atom selected from nitrogen, phosphorus, arsenic, oxygen, sulfur and carbon that includes an unshared electron pair.

The ligand may be a monodentate ligand, which includes only one atom bearing an unshared electron pair that may interact with the nickel. Exemplary monodentate ligands may include, but are not limited to, monophosphines, such as triarylphosphines, such as triphenylphosphine and substituted versions thereof (e.g., ortho-, meta- and para-substituted triphenylphosphines). Other exemplary monodentate ligands may include, but are not limited to, N-heterocyclic carbene ligands, such as, for example, 1,3-bis(2,6-diisopropylphenyl)imidazol-2-ylidene.

In some embodiments, the ligand may be a bidentate or “chelating” ligand, i.e., a ligand comprising two atoms bearing unshared electron pairs, with a spatial relationship therebetween, such that the atoms are capable of interacting simultaneously with the nickel atom or ion. For example, a chelating ligand may be a diamine, aminoalcohol, or a bis-phosphine.

A ligand may be included in a reaction mixture in an amount of about 2 mol % to about 20 mol %. For example, a ligand compound may be included in a reaction mixture in an amount of about 1, 2, 3, 4, 5, 6, 7, 8, 9, 10, 11, 12, 13, 14, 15, 16, 17, 18, 19, or 20 mol %. In some embodiments, a ligand may be included in a reaction mixture in an amount of about double the amount of the nickel-containing compound.

Together, the nickel-containing compound and one or more molecules of the ligand may interact to form a metal-ligand complex, which may serve as a catalyst for the reaction.

e. Reaction Conditions

The reaction mixture may further comprise a solvent. Any suitable solvent that is compatible with the components of the reaction mixture may be used. Suitably, a solvent will be selected such that the compounds of formula (II) and (III) will be at least partially soluble (or fully soluble), and will allow the reaction mixture to be heated to a temperature sufficient for the reaction to produce a compound of formula (I). Exemplary solvents include, but are not limited to: ethers such as diethyl ether, dibutyl ether, 1,2-dimethoxyethane, diglyme, t-butyl methyl ether, tetrahydrofuran, dioxane and the like; halogenated solvents such as chloroform, dichloromethane, dichloroethane, trifluorotoluene, chlorobenzene and the like; aliphatic or aromatic hydrocarbon solvents such as benzene, xylene, toluene, hexane, pentane and the like; esters and ketones such as ethyl acetate, acetone, 2-butanone and the like; polar aprotic solvents such as acetonitrile, dimethylformamide, dimethylsulfoxide and the like; or any combination of two or more solvents. For example, a suitable solvent is toluene.

The reaction may be conducted in a solution in which the concentration of the compound of formula (II) is from about 0.10 M to about 1.0 M, e.g., about 0.10 M, 0.15 M, 0.20 M, 0.25 M, 0.30 M, 0.35 M, 0.40 M, 0.45 M, 0.50 M, 0.55 M, 0.60 M, 0.65 M, 0.70 M, 0.75 M, 0.80 M, 0.85 M, 0.90 M, 0.95 M, or 1.0 M.

The solvent and/or the reaction mixture may be substantially anhydrous, i.e. may be substantially free of water. In some embodiments, the solvent and/or the reaction mixture may comprise less than about 10 wt. %, 9.5 wt. %, 9.0 wt. %, 8.5 wt. %, 8.0 wt. %, 7.5 wt. %, 7.0 wt. %, 6.5 wt. %, 6.0 wt. %, 5.5 wt. %, 5.0 wt. %, 4.5 wt. %, 4.0 wt. %, 3.5 wt. %, 3.0 wt. %, 2.5 wt. %, 2.0 wt. %, 1.5 wt. %, 1.0 wt. %, 0.95 wt. %, 0.90 wt. %, 0.85 wt. %, 0.80 wt. %, 0.75 wt. %, 0.70 wt. %, 0.65 wt. %, 0.60 wt. %, 0.55 wt. %, 0.50 wt. %, 0.45 wt. %, 0.40 wt. %, 0.35 wt. %, 0.30 wt. %, 0.25 wt. %, 0.20 wt. %, 0.15 wt. %, 0.10 wt. %, 0.09 wt. %, 0.08 wt. %, 0.07 wt. %, 0.06 wt. %, 0.05 wt. %, 0.04 wt. %, 0.03 wt. %, 0.02 wt. % or 0.01 wt. % water.

The method of synthesizing the compound of formula (I) may further comprise heating the reaction mixture. For example, the reaction mixture may be heated to a temperature of about 25° C. to about 100° C., or about 40° C. to about 80° C., e.g., to about 25° C., about 30° C., about 35° C., about 40° C., about 45° C., about 50° C., about 55° C., about 60° C., about 65° C., about 70° C., about 75° C., about 80° C., about 85° C., about 90° C., about 95° C., or about 100° C.

Other components may also be added to the reaction mixture, such as an acid, a base or a salt.

The method of synthesizing the compound of formula (I) may further comprise stirring the reaction mixture. For example, the reaction mixture may be stirred using a magnetic stirring bar, or an overhead mixer.

The reaction mixture may be contained within any suitable reaction vessel, such as a vial, flask, beaker, tube (e.g., a sealed tube), or the like. In some embodiments, the reaction vessel may be suitably dry, e.g., the reaction vessel may be dried in an oven and/or under vacuum.

The reaction mixture may further comprise an inert atmosphere. For example, a reaction vessel comprising the reaction mixture may consist essentially of an inert gas such as nitrogen, argon, or a mixture thereof. In some embodiments, an inert atmosphere comprises dioxygen (O₂) in an amount of less than about 1000 ppm, 900 ppm, 800 ppm, 700 ppm, 600 ppm, 500 ppm, 400 ppm, 300 ppm, 200 ppm, 100 ppm, 95 ppm, 90 ppm, 85 ppm, 80 ppm, 75 ppm, 70 ppm, 65 ppm, 60 ppm, 55 ppm, 50 ppm, 45 ppm, 40 ppm, 35 ppm, 30 ppm, 25 ppm, 20 ppm, 15 ppm, 10 ppm, 9 ppm, 8 ppm, 7 ppm, 6 ppm, 5 ppm, 4 ppm, 3 ppm, 2 ppm or 1 ppm O₂.

The method may comprise incubating, stirring and/or heating the reaction mixture for a period of time sufficient to form a compound of formula (I). For example, the reaction mixture may be incubated, stirred and/or heated for about 1 hour to about 12 hours, or about 2 hours to about 10 hours. For example, the reaction mixture may be incubated, stirred and/or heated for about 1 hour, 1.5 hours, 2 hours, 2.5 hours, 3.0 hours, 3.5 hours, 4.0 hours, 4.5 hours, 5.0 hours, 5.5 hours, 6.0 hours, 6.5 hours, 7.0 hours, 7.5 hours, 8.0 hours, 8.5 hours, 9.0 hours, 9.5 hours, 10 hours, 10.5 hours, 11 hours, 11.5 hours or 12 hours.

The method may provide a compound of formula (I) in a yield of about 50% to about 100%, e.g., about 60% to about 99%. For example, the method may provide a compound of formula (I) in about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% yield.

f. Optional Additional Method Steps

Methods of synthesizing compounds of formula (I) may optionally further include additional process steps. For example, the method may further comprise the step of purifying the compound of formula (I) from the reaction mixture. For example, the reaction mixture may be directly subjected to column chromatography (e.g., flash column chromatography) on a solid phase such as silica gel. The reaction mixture may alternatively be purified using other forms of chromatography, such as high pressure liquid chromatography (HPLC). The reaction mixture may be concentrated or the solvent may be removed prior to purification.

In some embodiments in which R¹ is a nitrogen protecting group, the method may further comprise the step of removing the nitrogen protecting group. In such methods, the protecting group may be removed using any suitable method that is capable of removing the protecting group. For example, in embodiments in which R¹ is a tert-butyloxycarbonyl (Boc) protecting group, the method may further comprise the step of reacting the compound of formula (I) with an acid (e.g., a strong acid such as hydrochloric acid or trifluoroacetic acid). In some embodiments in which R¹ is a p-toluenesulfonyl (tosyl, Ts) protecting group, the method may further comprise the step of reacting the compound of formula (I) with an acid (e.g., a strong acid such as hydrobromic acid or sulfuric acid), or a reducing agent (e.g., sodium or sodium amalgam).

Following preparation of the compound of formula (I), the carbonyl moiety may be reduced to the alcohol. For example, the method may further comprise the step of reacting the compound of formula (I) with a reducing agent (e.g., sodium borohydride or lithium aluminum hydride), to yield a hydroxylated piperidine compound.

g. Product

The product of the reaction is a compound of formula (I), as described above. The product of the reaction may be evaluated using a number of techniques. For example, compounds may be subjected to structural characterization using, for example, nuclear magnetic resonance (NMR) spectroscopy, mass spectrometry (MS), and infrared spectroscopy (IR). Reactions may be monitored for formation of a product using techniques such as gas chromatography (GC) or thin-layer chromatography (TLC). Purified products may be confirmed using elemental analysis (EA).

In methods for which a compound of formula (III) includes two R³ groups that are different, a reaction may proceed regioselectively. For example, the reaction may produce two products in a ratio of about 99:1 to about 80:20, e.g., about 99.9:0.1, 99:1, 98:2, 97:3, 96:4, 95:5, 94:6, 93:7, 92:8, 91:9, 90:10, 89:11, 8:12, 87:13, 86:14, 85:15, 84:16, 83:17, 82:18, 81:19 or 80:20. Exemplary two products 1 and 2 are illustrated in Scheme 1.

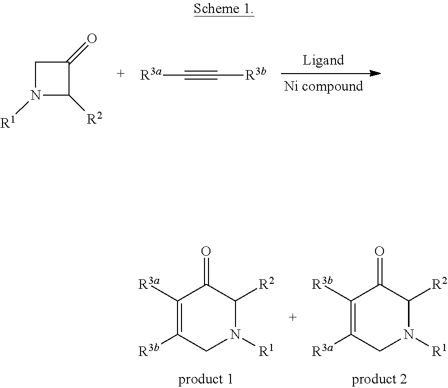

Products 1 and 2 may be separated using techniques known to those skilled in the art, such as chromatography (e.g., column chromatography such as flash column chromatography or HPLC).

### 3. COMPOUNDS

This disclosure also provides compounds of formula (I), which may be prepared by methods described herein and/or included in pharmaceutical compositions described herein. A compound may have the following formula (I):

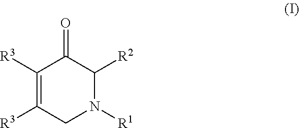

wherein:

R¹ is selected from the group consisting of hydrogen, alkyl or a nitrogen protecting group; and

R² is selected from the group consisting of hydrogen, alkyl, alkenyl, alkynyl, cycloalkyl, heterocyclyl, aryl, heteroaryl, silyl and stannyl, any of which may be optionally substituted; and

each R³ is independently selected from the group consisting of hydrogen, alkyl, alkenyl, alkynyl, cycloalkyl, heterocyclyl, aryl, heteroaryl, silyl and stannyl, any of which may be optionally substituted, or both R³ are taken together with the atoms to which they are attached to form an optionally substituted ring.

In some embodiments, R¹ is alkyl. In some embodiments, R¹ is a nitrogen protecting group, such as, e.g., tert-butyloxycarbonyl (Boc) or tosyl (Ts).

In some embodiments, R² is hydrogen. In some embodiments, R² is a substituted alkyl group such as an arylalkyl group.

In some embodiments, the two R³ groups are the same. In some embodiments, the two R³ groups are different.

In some embodiments, each R³ is independently selected from the group consisting of hydrogen, C₁-C₆ alkyl (e.g., C₁-C₄ alkyl such as methyl, ethyl, isopropyl, n-propyl and tert-butyl), C₂-C₆ alkenyl (e.g., C₂-C₄ alkenyl such as propenyl, such as prop-1-en-2-yl), silyl (e.g., trimethylsilyl), aryl (e.g., phenyl) including substituted aryl groups (e.g., phenyl substituted with alkoxy such as methoxy, or haloalkyl such as trifluoromethyl), heteroaryl (e.g., thienyl or furanyl) including substituted heteroaryl groups, and stannyl (e.g., tributylstannyl).

In some embodiments, both R³ are taken together with the atoms to which they are attached to form an optionally substituted ring. In some embodiments, both R³ are taken together with the atoms to which they are attached to form an unsubstituted ring. The ring may include at least 8 atoms. The ring may include up to 12 atoms.

a. Salt Forms

A compound of formula (I) may be in the form of a salt, e.g., a pharmaceutically acceptable salt. The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts may be prepared in situ during the final isolation and purification of the compounds or separately by reacting a compound with a suitable acid or base, depending on the particular substituents found on the compounds described herein. Neutral forms of the compounds may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of this disclosure. Examples of pharmaceutically acceptable salts are discussed in Berge et al, 1977, “Pharmaceutically Acceptable Salts.” J. Pharm. Sci. Vol. 66, pp. 1-19.

Representative acid addition salts may be prepared using various suitable acids for example, including, but not limited to, acetic, adipic, alginic, citric, aspartic, benzoic, benzenesulfonic, butyric, camphoric, camphorsulfonic, carbonic, digluconic, glycerophosphoric, heptanoic, hexanoic, fumaric, hydrochloric, hydrobromic, hydroiodic, 2-hydroxyethansulfonic (isethionic), lactic, maleic, methanesulfonic, nicotinic, 2-naphthalenesulfonic, oxalic, pamoic, pectinic, persulfuric, 3-phenylpropionic, picric, pivalic, propionic, succinic, sulfuric, tartaric, thiocyanic, phosphoric, glutamatic, p-toluenesulfonic, and undecanoic acids.

Particular examples of acids which may be employed to form pharmaceutically acceptable acid addition salts include such inorganic acids as hydrochloric acid, hydrobromic acid, sulfuric acid and phosphoric acid and such organic acids as oxalic acid, maleic acid, succinic acid, tartaric acid, and citric acid.

Basic addition salts may be prepared in situ during the final isolation and purification of compounds by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

Also, the basic nitrogen-containing groups may be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Unless otherwise specified, a reference to a particular compound also includes salt forms thereof

b. Isomers

Certain compounds may exist in one or more particular geometric, optical, enantiomeric, diastereomeric, epimeric, atropic, stereoisomer, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; a- and β-forms; axial and equatorial forms; boat-, chair-, twist-, envelope-, and half chair-forms; and combinations thereof, hereinafter collectively referred to as “isomers” (or “isomeric forms”).

In one embodiment, a compound described herein may be an enantiomerically enriched isomer of a stereoisomer described herein, or a method of synthesizing a compound may produce an enantiomerically enriched isomer of a stereoisomer described herein. For example, the compound may have an enantiomeric excess of at least about 10%, 15%, 20%, 25%, 30%, 35%, 40%, 45%, 50%, 55%, 60%, 65%, 70%, 75%, 80%, 85%, 90%, 95%, 96%, 97%, 98%, or 99%. Enantiomer, when used herein, refers to either of a pair of chemical compounds whose molecular structures have a mirror-image relationship to each other.

In one embodiment, a preparation of a compound disclosed herein is enriched for an isomer of the compound having a selected stereochemistry, e.g., R or S, corresponding to a selected stereocenter. For example, the compound has a purity corresponding to a compound having a selected stereochemistry of a selected stereocenter of at least about 60%, 65%, 70%, 75%, 80%, 85%, 90%, 95%, 96%, 97%, 98%, or 99%.

In one embodiment, a composition described herein includes a preparation of a compound disclosed herein that is enriched for a structure or structures having a selected stereochemistry, e.g., R or S, at a selected stereocenter. Exemplary R/S configurations may be those provided in an example described herein.

An “enriched preparation,” as used herein, is enriched for a selected stereoconfiguration of one, two, three or more selected stereocenters within the subject compound. Exemplary selected stereocenters and exemplary stereoconfigurations thereof may be selected from those provided herein, e.g., in an example described herein. By enriched is meant at least 60%, e.g., of the molecules of compound in the preparation have a selected stereochemistry of a selected stereocenter. In an embodiment it is at least 65%, 70%, 75%, 80%, 85%, 90%, 95%, 96%, 97%, 98%, or 99%. Enriched refers to the level of a subject molecule(s) and does not connote a process limitation unless specified.

Compounds may be prepared in racemic form or as individual enantiomers or diastereomers by either stereospecific synthesis or by resolution. The compounds may, for example, be resolved into their component enantiomers or diastereomers by standard techniques, such as the formation of stereoisomeric pairs by salt formation with an optically active base, followed by fractional crystallization and regeneration of the free acid. The compounds may also be resolved by formation of stereoisomeric esters or amides, followed by chromatographic separation and removal of the chiral auxiliary. Alternatively, the compounds may be resolved using a chiral HPLC column. The enantiomers also may be obtained from kinetic resolution of the racemate of corresponding esters using lipase enzymes.

Except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₃-alkyl or propyl includes n-propyl and iso-propyl; C₄-alkyl or butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol, imine/enamine, amide/imino alcohol, amidine/amidine, nitroso/oxime, thioketone/enethiol, N-nitroso/hydroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

### 3. PHARMACEUTICAL COMPOSITIONS

The disclosure also provides pharmaceutical compositions comprising a compound of formula (I), and a pharmaceutically acceptable carrier. The term “pharmaceutically acceptable carrier,” as used herein, means a non-toxic, inert solid, semi-solid or liquid filler, diluent, encapsulating material or formulation auxiliary of any type. Some examples of materials which may serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants may also be present in the composition, according to the judgment of one skilled in the art of formulations.

The pharmaceutical compositions may be administered to subjects (e.g., humans and other mammals) orally, rectally, parenterally, intracisternally, intravaginally, intraperitoneally, topically (as by powders, ointments or drops), bucally or as an oral or nasal spray. The term “parenterally,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like, and suitable mixtures thereof), vegetable oils (such as olive oil) and injectable organic esters such as ethyl oleate, or suitable mixtures thereof. Suitable fluidity of the composition may be maintained, for example, by the use of a coating such as lecithin, by the maintenance of the required particle size in the case of dispersions, and by the use of surfactants.

These compositions may also contain adjuvants, such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It also may be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug may depend upon its rate of dissolution, which, in turn, may depend upon crystal size and crystalline form. Alternatively, a parenterally administered drug form may be administered by dissolving or suspending the drug in an oil vehicle.

Suspensions, in addition to the active compounds, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds may be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Injectable depot forms are made by forming microencapsulated matrices of the drug in biodegradable polymers such as polylactide-polyglycolide. Depending upon the ratio of drug to polymer and the nature of the particular polymer employed, the rate of drug release may be controlled. Examples of other biodegradable polymers include poly(orthoesters) and poly(anhydrides) Depot injectable formulations also are prepared by entrapping the drug in liposomes or microemulsions which are compatible with body tissues.

The injectable formulations may be sterilized, for example, by filtration through a bacterial-retaining filter or by incorporating sterilizing agents in the form of sterile solid compositions which may be dissolved or dispersed in sterile water or other sterile injectable medium just prior to use.

Injectable preparations, for example, sterile injectable aqueous or oleaginous suspensions may be formulated according to the known art using suitable dispersing or wetting agents, suspending agents and the like. The sterile injectable preparation also may be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid are used in the preparation of injectables.

Solid dosage forms for oral administration include, but are not limited to, capsules, tablets, pills, powders, and granules. In such solid dosage forms, one or more compounds is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols.

The solid dosage forms of tablets, dragees, capsules, pills, and granules may be prepared with coatings and shells such as enteric coatings and other coatings well-known in the pharmaceutical formulating art. They optionally may contain opacifying agents and also may be of a composition that they release the active ingredient(s) only, or preferentially, in a certain part of the intestinal tract in a delayed manner. Examples of materials useful for delaying release of the active agent may include polymeric substances and waxes.

Compositions for rectal or vaginal administration are preferably suppositories which may be prepared by mixing the compounds with suitable non-irritating carriers such as cocoa butter, polyethylene glycol or a suppository wax which are solid at ambient temperature but liquid at body temperature and therefore melt in the rectum or vaginal cavity and release the active compound.

Liquid dosage forms for oral administration may include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions may also include adjuvants such as wetting agents, emulsifying and suspending agents, sweetening, flavoring, and perfuming agents.

Suspensions, in addition to the active compounds, may contain suspending agents, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds may be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

Dosage forms for topical or transdermal administration of a compound include ointments, pastes, creams, lotions, gels, powders, solutions, sprays, inhalants or patches. A desired compound is admixed under sterile conditions with a pharmaceutically acceptable carrier and any needed preservatives or buffers as may be required. Ophthalmic formulation, eardrops, eye ointments, powders and solutions are also contemplated as being within the scope of this disclosure.

The ointments, pastes, creams and gels may contain, in addition to an active compound, animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to the compounds, lactose, talc, silicic acid, aluminum hydroxide, calcium silicates and polyamide powder, or mixtures of these substances. Sprays additionally may contain customary propellants such as chlorofluorohydrocarbons.

Compounds also may be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds, stabilizers, preservatives, and the like. The preferred lipids are the natural and synthetic phospholipids and phosphatidylcholines (lecithins) used separately or together. Methods to form liposomes are known in the art. See, for example, Prescott, Ed., Methods in Cell Biology, Volume XIV, Academic Press, New York, N.Y., (1976), p 33 et seq.

Dosage forms for topical administration of a compound, described herein, include powders, sprays, ointments and inhalants. The active compound is mixed under sterile conditions with a pharmaceutically acceptable carrier and any needed preservatives, buffers or propellants. Ophthalmic formulations, eye ointments, powders and solutions are also contemplated as being within the scope of this disclosure. Aqueous liquid compositions may also be useful.

It will be readily apparent to those skilled in the art that other suitable modifications and adaptations of the compounds and methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples which are merely intended only to illustrate some aspects and embodiments of the disclosure, and should not be viewed as limiting to the scope of the disclosure. The disclosures of all journal references, U.S. patents and publications referred to herein are hereby incorporated by reference in their entireties.

### EXAMPLES

### General Experimental and Analytical Details

All reactions were conducted under an atmosphere of N₂ using standard Schlenk techniques or in a N₂ filled glove-box unless otherwise noted. Toluene was dried over neutral alumina under N₂ using a Grubbs type solvent purification system. Ni(COD)₂ was purchased from Strem and used without further purification. 1-Boc-3-azetidinone was purchased from Sigma-Aldrich and used as received. All other reagents were purchased from commercial suppliers and used without further purification unless otherwise noted.

¹H and ¹³C Nuclear Magnetic Resonance spectra of pure compounds were acquired at 400 and 100 MHz, respectively unless otherwise noted. All spectra are referenced to a singlet at 7.27 ppm for ¹H and to the center line of a triplet at 77.23 ppm for ¹³C. The abbreviations s, d, dd, dt, dq, t, q, and quint stand for singlet, doublet, doublet of doublets, doublet of triplets, doublet of quartets, triplet, quartet, and quintet, in that order. All ¹³C NMR spectra were proton decoupled. Gas Chromatography was performed using the following conditions: initial oven temperature: 100° C.; temperature ramp rate 50° C./min.; final temperature: 300° C. held for 7 minutes; detector temperature: 250° C.

### Example 1

### Reaction Screening

Reactions were screened to determine optimal ligands and protecting groups. To screen protecting groups, reactions were performed as shown in Scheme 2.

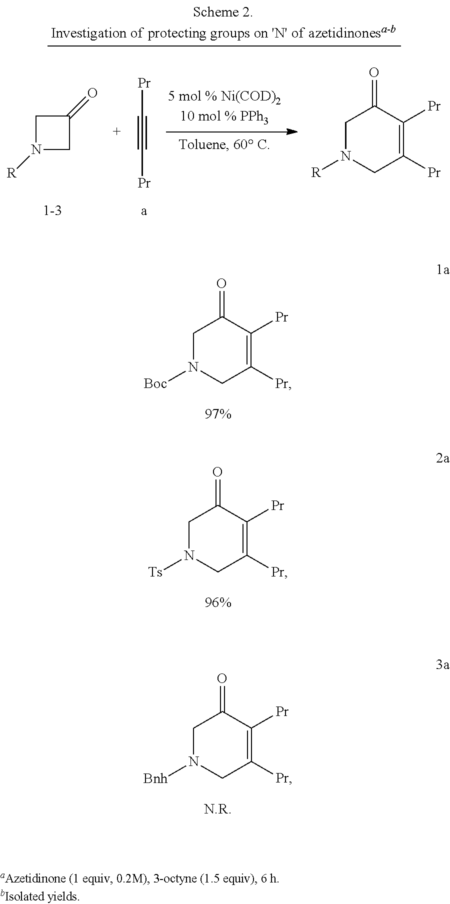

The tert-butoxycarbonyl- and tosyl-azetidinones were converted to piperidone products in good to excellent yields. Under these reaction conditions, the use of a benzhydryl protecting group did not lead to the desired cycloadduct.

In a nitrogen filled glovebox, stock solution (0.1M) of 1-Boc-3-azetidinone (i.e. tert-butyl 3-oxoazetidine-1-carboxylate) (1 equiv) in toluene was prepared along with decane as an internal standard in a clean and pre-dried scintillation vial. The stock solution of oct-4-yne (1.5 equiv) in toluene was also prepared in a separate vial. In separate vials, stock solutions of catalyst were prepared by mixing Ni(cod)₂ and ligands. 10 mol % catalyst was added to the vial containing the azetidinone and the alkyne. The vials were taken out of the glove box and stirred @ 60° C. overnight, after which all the reaction vials were opened to air and then analyzed by GC. Triphenylphosphine was found to be optimal.

### Example 2

### Compound Synthesis

### General Procedure ‘A’ for Cycloaddition

In a nitrogen-filled glove box, 5 mol % catalyst solution (prepared from Ni(cod)₂ and PPh₃ in 1:2 molar ratio in toluene) was added to the vial containing the appropriate azetidinone (1 equiv, 0.1 M or 0.2M) and the appropriate alkyne (1.5 equiv) in toluene. The vial was taken out of the glove box and stirred @ 60° C. for 6 h, opened to air, concentrated in vacuo, and purified by silica gel flash column chromatography.

### General Procedure ‘B’ for Cycloaddition

In a nitrogen-filled glove box, 5 mol % catalyst solution (prepared from Ni(cod)₂ and PPh₃ in 1:2 molar ratio in toluene) was added to the vial (fitted with a PTFE septum) containing the appropriate azetidinone (1 equiv, 0.1 M). The vial was taken out of the glove box and stirred @ 100° C. Then solution of the appropriate alkyne (3.0 equiv) in toluene was added to the vial containing the azetidinone over a period of 2 h and stirred for another 4 h @ 100° C., opened to air, concentrated in vacuo, and purified by silica gel flash column chromatography.

### Tert-butyl 5-oxo-3,4-dipropyl-5,6-dihydropyridine-1(2H)-carboxylate (1a)

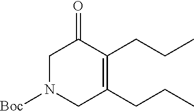 [SMILES: CCCC1=C(CCC)C(=O)CN(C(=O)OC(C)(C)C)C1]

The general procedure ‘A’ was used with 59.7 mg (0.35 mmol, 0.1 M) of 1-Boc-3-azetidinone, 57.6 mg (0.52 mmol) of oct-4-yne, and 5 mol % of catalyst in toluene. The reaction mixture was purified via flash column chromatography using 15% ethyl acetate in hexanes to afford 95.1 mg of the title compound 1a as colorless oil, 97% yield. ¹H NMR (400 MHz, CDCl₃): δ (ppm) 4.10 (br, s, 2H), 4.01 (s, 2H), 2.24 (t, 4H, J=6 Hz), 1.53 (sextet, 2H, J=6 Hz), 1.44 (s, 9H), 1.33 (sextet, 2H, J=6 Hz), 0.97 (t, 3H, J=6 Hz), 0.89 (t, 3H, J=6 Hz). ¹³C NMR (100 MHz, CDCl₃): δ (ppm) 193.4, 156.2 (br), 154.3, 134.4, 80.8, 51.7 (br), 45.9 (br), 34.4, 28.5, 26.8, 22.7, 21.6, 14.44, 14.40. IR (CH₂Cl₂, cm⁻¹): 2964, 2873, 1704, 1677, 1420, 1368, 1242, 1168, 1136, 905, 769. HRMS (ESI) calcd for C16H27NO3Na [M+Na]+ 304.1889, found 304.1892.

### 4,5-Dipropyl-1-tosyl-1,6-dihydropyridin-3(2H)-one (2a)

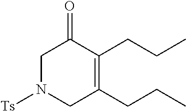 [SMILES: CCCC1=C(CCC)C(=O)CN(S(=O)(=O)c2ccc(C)cc2)C1]

The general procedure ‘A’ was used with 20.4 mg (0.09 mmol, 0.1 M) of 1-tosyl-3-azetidinone (i.e. 3-oxoazetidin-1-yl 4-methylbenzenesulfonate), 15.0 mg (0.14 mmol) of oct-4-yne, and 5 mol % of catalyst in toluene. The reaction mixture was purified via flash column chromatography using 15% ethyl acetate in hexanes to afford 28.8 mg of the title compound 2a as colorless oil, 96% yield. ¹H NMR (400 MHz, CDCl₃): δ (ppm) 7.64 (d, 2H, J=6 Hz), 7.33 (d, 2H, J=6 Hz), 3.86 (s, 2HHz), 0.97 (t, 3H, J=6 Hz), 0.85 (t, 3H, J=6 Hz). ¹³C NMR (100 MHz, CDCl₃): δ (ppm) 191.4, 153.9, 144.4, 135.0, 133.1, 130.1, 127.9, 52.7, 48.0, 34.6, 26.7, 22.6, 21.7, 21.5, 14.5, 14.4. IR (CH₂Cl₂, cm⁻¹): 2962, 2932, 2872, 1676, 1494, 1351, 1166, 1090, 1039, 963, 839, 815, 673, 582, 547. HRMS (ESI) calcd for C18H25NO3NaS [M+Na]+ 358.1477, found 358.1443.

### Tert-butyl 3,4-dimethyl-5-oxo-5,6-dihydropyridine-1(2H)-carboxylate (1b)

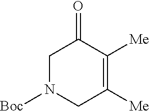 [SMILES: CC1=C(C)C(=O)CN(C(=O)OC(C)(C)C)C1]

The general procedure ‘A’ was used with 28.1 mg (0.16 mmol, 0.1 M) of 1-Boc-3-azetidinone, 13.3 mg (0.25 mmol) of 2-butyne, and 5 mol % of catalyst in toluene. The reaction mixture was purified via flash column chromatography using 15-20% ethyl acetate in hexanes to afford 35.0 mg of the title compound 1b as colorless oil, 95% yield. ¹H NMR (400 MHz, CDCl₃): δ (ppm) 4.08 (s, 2H), 4.01 (s, 2H), 1.90 (s, 3H), 1.75 (s, 3H), 1.41 (s, 9H). ¹³C NMR (100 MHz, CDCl₃): δ (ppm) 193.1, 154.1, 152.5 (br), 129.9, 80.8, 51.2 (br), 47.3 (br), 28.4, 18.4, 10.2. IR (CH₂Cl₂, cm⁻¹): 2976, 2930, 1701, 1678, 1420, 1366, 1323, 1282, 1243, 1172, 1136, 897, 856, 769. HRMS (ESI) calcd for C12H19NO3Na [M+Na]+ 248.1263, found 248.1256.

### Tert-butyl 3-(tert-butyl)-4-methyl-5-oxo-5,6-dihydropyridine-1(2H)-carboxylate (1c)

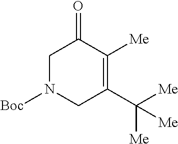 [SMILES: CC1=C(C(C)(C)C)CN(C(=O)OC(C)(C)C)CC1=O]

The general procedure ‘A’ was used with 28.0 mg (0.16 mmol, 0.1 M) of 1-Boc-3-azetidinone, 23.59 mg (0.25 mmol) of 4,4-dimethylpent-2-yne, and 5 mol % of catalyst in toluene. The reaction mixture was purified via flash column chromatography using 15% ethyl acetate in hexanes to afford 40.6 mg of the title compound 1c as colorless oil, 93% yield. ¹H NMR (400 MHz, CDCl₃): δ (ppm) 4.21 (s, 2H), 4.01 (s, 2H), 1.96 (t, 3H, J=3 Hz), 1.46 (s, 9H), 1.28 (s, 9H). ¹³C NMR (100 MHz, CDCl₃): δ (ppm) 195.0, 162.6, 154.4, 130.5, 80.9, 51.0 (br), 44.9 (br), 29.2, 28.5, 13.4. IR (CH₂Cl₂, cm⁻¹): 2975, 1679, 1606, 1429, 1370, 1252, 1167, 1128, 1074, 1040, 911, 856, 770. HRMS (ESI) calcd for C15H25NO3Na [M+Na]+ 290.1732, found 290.1738.

### 5-(Tert-butyl)-4-methyl-1-tosyl-1,6-dihydropyridin-3(2H)-one (2c)

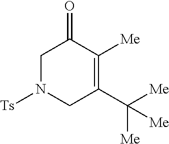 [SMILES: CC1=C(C(C)(C)C)CN(S(=O)(=O)c2ccc(C)cc2)CC1=O]

The general procedure ‘A’ was used with 21.1 mg (0.09 mmol, 0.1 M) of 1-tosyl-3-azetidinone (i.e. 3-oxoazetidin-1-yl 4-methylbenzenesulfonate), 13.51 mg (0.14 mmol) of 4,4-dimethylpent-2-yne, and 5 mol % of catalyst in toluene. The reaction mixture was purified via flash column chromatography using 20% ethyl acetate in hexanes to afford the title compound 2c as colorless solid, 77% yield. ¹H NMR (400 MHz, CDCl₃): δ (ppm) 7.64 (d, 2H, J=8 Hz), 7.33 (d, 2H, J=8 Hz), 3.95 (s, 2H), 3.72 (s, 2H), 2.42 (s, 3H), 1.81 (t, 3H, J=1.6 Hz), 1.22 (s, 9H). ¹³C NMR (100 MHz, CDCl₃): δ (ppm) 192.8, 159.7, 144.3, 133.3, 131.2, 130.1, 127.9, 52.2, 47.0, 37.1, 29.1, 21.7, 13.2. IR (CH₂Cl₂, cm⁻¹): 2969, 2868, 2824, 1674, 1598, 1444, 1348, 1165, 1089, 1036, 1007, 959, 665, 580, 547. HRMS (ESI) calcd for C17H23NO3NaS [M+Na]+ 344.1296, found 344.1307. Tert-butyl 3-(tert-butyl)-5-oxo-5,6-dihydropyridine-1(2H)-carboxylate (1d)

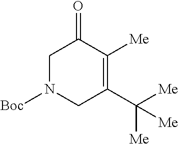 [SMILES: CC1=C(C(C)(C)C)CN(C(=O)OC(C)(C)C)CC1=O]

The general procedure ‘B’ was used with 23.0 mg (0.13 mmol, 0.2 M) of 1-Boc-3-azetidinone, 33.10 mg (0.40 mmol) of 3,3-dimethylbut-1-yne, and 5 mol % of catalyst in toluene. The reaction mixture was purified via flash column chromatography using 20% ethyl acetate in hexanes to afford the title compound 1d as colorless oil, 71% yield. ¹H NMR (400 MHz, CDCl₃): δ (ppm) 6.04 (t, 1H, J=1.6 Hz), 4.22 (s, 2H), 4.02 (s, 2H), 1.46 (s, 9H), 1.18 (s, 9H). ¹³C NMR (100 MHz, CDCl₃): δ (ppm) 194.8, 154.4, 121.7, 81.0, 51.4 (br), 42.8 (br), 36.3, 28.5. IR (CH₂Cl₂, cm⁻¹): 2971, 2875, 1701, 1683, 1620, 1477, 1418, 1367, 1236, 1165, 1112, 903, 884, 854, 767. HRMS (ESI) calcd for C14H23NO3Na [M+Na]+ 276.1576, found 276.1581.

### Tert-butyl 3-methyl-5-oxo-4-(trimethylsilyl)-5,6-dihydropyridine-1(2H)-carboxylate (1e)

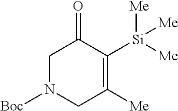 [SMILES: CC1=C([Si](C)(C)C)C(=O)CN(C(=O)OC(C)(C)C)C1]

The general procedure ‘A’ was used with 41.9 mg (0.25 mmol, 0.1 M) of 1-Boc-3-azetidinone, 41.2 mg (0.37 mmol) of trimethyl(prop-1-ynyl)silane, and 5 mol % of catalyst in toluene. The reaction mixture was purified via flash column chromatography using 10-15% ethyl acetate in hexanes to afford 63.8 mg (55 mg major regioisomer, 4.4 mg minor regioisomer, and 4.4 mg mixture of both regioisomers) of the title compound 1e as colorless oil, 92% yield. ¹H NMR (400 MHz, CDCl₃): (major isomer) δ (ppm) 4.03 (s, 2H), 3.94 (s, 2H), 2.03 (s, 3H), 1.46 (s, 9H), 0.23 (s, 9H). ¹³C NMR (100 MHz, CDCl₃): δ (ppm) 197.1, 166.2 (br), 154.3, 135.5, 80.9, 51.3 (br), 48.3 (br), 28.5, 21.8, 1.3. IR (CH₂Cl₂, cm⁻¹): 2976, 2901, 2824, 1700, 1664, 1596, 1477, 1418, 1365, 1245, 1161, 1116, 1054, 945, 899, 845, 765, 691. HRMS (ESI) calcd for C14H25NO3NaSi [M+Na]+ 306.1501, found 306.1506. ¹H NMR (300 MHz, CDCl₃): (minor isomer) δ (ppm) 4.22 (s, 2H), 4.08 (s, 2H), 1.94 (t, 3H, J=2.1 Hz), 1.48 (s, 9H), 0.28 (s, 9H).

### Tert-butyl 4-methyl-5-oxo-3-(tributylstannyl)-5,6-dihydropyridine-1(2H)-carboxylate (1f)

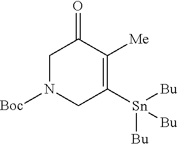

The general procedure ‘A’ was used with 23.1 mg (0.13 mmol, 0.1 M) of 1-Boc-3-azetidinone, 66.61 mg (0.20 mmol) of tributyl(prop-1-ynyl)stannane, and 5 mol % of catalyst in toluene (0.2M). The reaction mixture was purified via flash column chromatography using 5 to 10% ethyl acetate in hexanes to afford 60.6 mg of the title compound 1f as colorless oil, 89% yield. ¹H NMR (400 MHz, CDCl₃): δ (ppm) 4.06 (s, 2H), 4.00 (s, 2H), 2.01 (t, 3H, J=2.5 Hz), 1.47 (m, 16H), 1.30 (sextet, 6H, J=3 Hz), 1.09 (m, 1H), 1.01 (m, 5H), 0.94 (m, 1H), 0.88 (m, 5 Hz). ¹³C NMR (100 MHz, CDCl₃): δ (ppm) 197.1, 166.9 (br), 154.4, 139.4 (br), 80.9, 50.7 (br), 48.04 (br), 29.29, 28.54, 27.5, 23.66, 13.86, 11.74 (extra peaks are due to the coupling of Sn nucleus with alpha, beta, and/or gamma carbon). IR (CH₂Cl₂, cm⁻¹): 2957, 2926, 2854, 1704, 1658, 1601, 1416, 1368, 1240, 1160, 1109, 1076, 895, 771, 670, 597. HRMS (ESI) calcd for C23H43NO3NaSn [M+Na]+ 524.2163, found 524.2185.

### Tert-butyl 4-ethyl-5-oxo-3-(prop-1-en-2-yl)-5,6-dihydropyridine-1(2H)-carboxylate (1g)

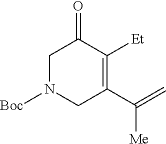 [SMILES: C=C(C)C1=C(CC)C(=O)CN(C(=O)OC(C)(C)C)C1]

The general procedure ‘A’ was used with 39.7 mg (0.23 mmol, 0.2 M) of 1-Boc-3-azetidinone, 32.75 mg (0.35 mmol) of 2-methylhex-1-en-3-yne, and 5 mol % of catalyst in toluene. The reaction mixture was purified via flash column chromatography using 15% ethyl acetate in hexanes to afford 40.6 mg of the title compound 1g as colorless oil, 87% yield. ¹H NMR (400 MHz, CDCl₃): δ (ppm) 5.13 (q, 1H, J=1.2 Hz), 4.86 (s, 1H), 4.15 (s, 2H), 4.06 (s, 2H), 2.27 (q, 2H, J=7.6 Hz), 1.93 (m, 3H), 1.46 (s, 9H), 0.97 (t, 3H, J=7.6 Hz). ¹³C NMR (100 MHz, CDCl₃): δ (ppm) 193.7, 156.5 (br), 154.3, 141.4, 134.7, 115.4, 81.0, 51.8 (br), 45.8 (br), 28.5, 22.1, 19.6, 14.5. IR (CH₂Cl₂, cm⁻¹): 3083, 2975, 2934, 2876, 1702, 1681, 1621, 1417, 1368, 1238, 1169, 1129, 905, 866, 768. HRMS (ESI) calcd for C15H23NO3Na [M+Na]+ 288.1576, found 288.1580.

### Tert-butyl 5-oxo-3,4-diphenyl-5,6-dihydropyridine-1(2H)-carboxylate (1h)

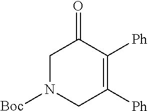 [SMILES: CC(C)(C)OC(=O)N1CC(=O)C(c2ccccc2)=C(c2ccccc2)C1]

The general procedure ‘B’ was used with 22.5 mg (0.13 mmol, 0.2 M) of 1-Boc-3-azetidinone, 70.27 mg (0.39 mmol) of 1,2-diphenylethyne, and 5 mol % of catalyst in toluene. The reaction mixture was purified via flash column chromatography using 20-30% ethyl acetate in hexanes to afford 36.3 mg of the title compound 1h as pale oil, 79% yield. ¹H NMR (400 MHz, CDCl₃): δ (ppm) 7.19 (m, 6H), 7.11 (m, 2H), 7.00 (m, 2H), 4.6 (2H), 4.33 (s, 2H), 1.54 (s, 9H). ¹³C NMR (100 MHz, CDCl₃): δ (ppm) 192.7, 154.4, 137.2, 136.1 (br), 133.8, 131.0, 129.0, 128.7, 128.4, 127.9, 127.5, 81.4, 52.1 (br), 47.9 (br), 28.6. IR (CH₂Cl₂, cm⁻¹): 3059, 2979, 2932, 1760, 1696, 1479, 1413, 1368, 1326, 1243, 1157, 1115, 993, 931, 858, 762, 737, 699. HRMS (ESI) calcd for C22H23NO3Na [M+Na]+ 372.1576, found 372.1583.

### Tert-butyl 4-methyl-5-oxo-3-phenyl-5,6-dihydropyridine-1(2H)-carboxylate (1i)

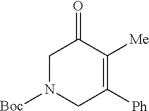 [SMILES: CC1=C(c2ccccc2)CN(C(=O)OC(C)(C)C)CC1=O]

The general procedure ‘B’ was used with 20.8 mg (0.12 mmol, 0.2 M) of 1-Boc-3-azetidinone, 42.34 mg (0.25 mmol) of prop-1-ynylbenzene, and 5 mol % of catalyst in toluene. The reaction mixture was purified via flash column chromatography using 15% ethyl acetate in hexanes to afford 28.2 mg of the title compound 1i as colorless oil, 81% yield. ¹H NMR (400 MHz, CDCl₃): δ (ppm) 7.42 (m, 3H), 7.28 (d, 2H, J=6.8 Hz), 4.41 (s, br, 2H), 4.20 (s, 2H), 1.78 (t, 3H, J=1.7 Hz), 1.49 (s, 9H). ¹³C NMR (100 MHz, CDCl₃): δ (ppm) 194.3, 154.3, 137.4, 130.8 (br), 129.1, 128.8, 128.2, 127.8, 81.1, 51.7 (br), 47.6 (br), 28.5, 12.3. IR (CH₂Cl₂, cm⁻¹): 3059, 2929, 1699, 1632, 1476, 1418, 1327, 1281, 1244, 1120, 1069, 1032, 1000, 897, 861, 765, 702, 623. HRMS (ESI) calcd for C17H21NO3Na [M+Na]+ 310.1419, found 310.1422.

### Tert-butyl 3-(4-methoxyphenyl)-4-methyl-5-oxo-5,6-dihydropyridine-1(2H)-carboxylate (1j)

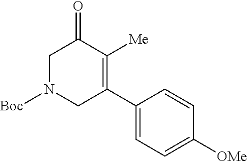 [SMILES: COc1ccc(C2=C(C)C(=O)CN(C(=O)OC(C)(C)C)C2)cc1]

The general procedure ‘B’ was used with 32.9 mg (0.19 mmol, 0.2 M) of 1-Boc-3-azetidinone, 78.48 mg (0.57 mmol) of 1-methoxy-4-(prop-1-ynyl)benzene, and 5 mol % of catalyst in toluene. The reaction mixture was purified via flash column chromatography using 20% ethyl acetate in hexanes to afford 48.5 mg of the title compound 1j as pale yellow oil, 74% yield. ¹H NMR (400 MHz, CDCl₃): δ (ppm) 7.23 (d, 2H, J=8.4 Hz), 6.94 (d, 2H, 8.8 Hz), 4.39 (s, 2H), 4.16 (s, 2H), 3.83 (s, 2H), 1.79 (t, 3H, J=1.8 Hz), 1.47 (s, 9H). ¹³C NMR (100 MHz, CDCl₃): δ (ppm) 194.4, 160.3, 154.3, 130.3, 129.6, 129.4, 55.5, 51.6 (br), 47.6 (br), 28.5, 12.5. IR (CH₂Cl₂, cm⁻¹): 2976, 2932, 2838, 1698, 1677, 1608, 1512, 1417, 1365, 1250, 1170, 1119, 1033, 945, 834, 768. HRMS (ESI) calcd for C18H23NO4Na [M+Na]+ 340.1525, found 340.1530.

### Tert-butyl 4-methyl-5-oxo-3-(4-(trifluoromethyl)phenyl)-5,6-dihydropyridine-1(2H)-carboxylate (1k)

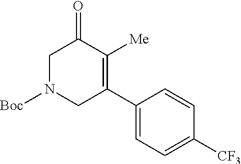 [SMILES: CC1=C(c2ccc(C(F)(F)F)cc2)CN(C(=O)OC(C)(C)C)CC1=O]

The general procedure ‘B’ was used with 39.1 mg (0.23 mmol, 0.2 M) of 1-Boc-3-azetidinone, 126.18 mg (0.68 mmol) of 1-(prop-1-ynyl)-4-(trifluoromethyl)benzene, and 5 mol % of catalyst in toluene. The reaction mixture was purified via flash column chromatography using 20% ethyl acetate in hexanes to afford the title compound 1k as colorless oil, 63% yield. ¹H NMR (400 MHz, CDCl₃): δ (ppm) 7.69 (d, 2H, J=8.4 Hz), 7.39 (d, 2H, J=8.0 Hz), 4.38 (s, 2H), 4.20 (s, 2H), 1.74 (t, 3H, J=2 Hz), 1.48 (s, 9H). ¹³C NMR (100 MHz, CDCl₃): δ (ppm) 193.9, 154.2, 141.0, 131.3 (t, J=35 Hz), 128.3, 125.9 (q, J=15.2 Hz), 125.3, 122.6, 81.4, 51.6, 47.5, 28.5, 12.2. IR (CH₂Cl₂, cm⁻¹): 2980, 2933, 1687, 1617, 1477, 1408, 1367, 1325, 1281, 1244, 1167, 1127, 1068, 1019, 998, 900, 844, 768, 686, 613. HRMS (ESI) calcd for C18H20NO3F3Na [M+Na]+ 378.1293, found 378.1292.

### Tert-butyl 5-oxo-3-phenyl-4-(trimethylsilyl)-5,6-dihydropyridine-1(2H)-carboxylate (1l)

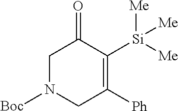 [SMILES: CC(C)(C)OC(=O)N1CC(=O)C([Si](C)(C)C)=C(c2ccccc2)C1]

The general procedure ‘B’ was used with 29.3 mg (0.17 mmol, 0.2 M) of 1-Boc-3-azetidinone, 89.47 mg (0.51 mmol) of trimethyl(phenylethynyl)silane, and 5 mol % of catalyst in toluene. The reaction mixture was purified via flash column chromatography using 10-15% ethyl acetate in hexanes to afford 48.5 mg of the title compound 1l as colorless oil, 82% yield. ¹H NMR (400 MHz, CDCl₃): δ (ppm) 7.41 (m, 3H), 7.28 (m, 2H), 4.32 (s, 2H), 4.09 (s, 2H), 1.51 (s, 9H), −0.12 (s, 9H). ¹³C NMR (100 MHz, CDCl₃): δ (ppm) 198.0, 168.6 (br), 154.4, 139.5, 137.7, 129.6, 128.6, 128.1, 81.1, 51.7 (br), 49.2 (br), 28.6, 0.5. IR (CH₂Cl₂, cm⁻¹): 2977, 2899, 1702, 1669, 1582, 1478, 1412, 1365, 1246, 1164, 1115, 1044, 1000, 939, 905, 845, 762, 700. HRMS (ESI) calcd for C19H27NO3NaSi [M+Na]+ 368.1658, found 368.1661.

### Tert-butyl 3-(furan-3-yl)-5-oxo-4-(trimethylsilyl)-5,6-dihydropyridine-1(2H)-carboxylate (1m)

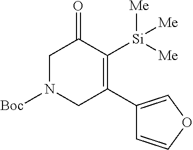 [SMILES: CC(C)(C)OC(=O)N1CC(=O)C([Si](C)(C)C)=C(c2ccoc2)C1]

The general procedure ‘B’ was used with 32.0 mg (0.18 mmol, 0.2 M) of 1-Boc-3-azetidinone, 92.1 mg (0.56 mmol) of (furan-3-ylethynyl)trimethylsilane, and 5 mol % of catalyst in toluene. The reaction mixture was purified via flash column chromatography using 10-15% ethyl acetate in hexanes to afford 51.1 mg of the title compound 1m as colorless oil, 82% yield. ¹H NMR (400 MHz, CDCl₃): δ (ppm) 7.47 (m, 2H), 6.48 (s, 1H), 4.24 (s, br, 2H), 4.06 (s, 2H), 1.49 (s, 9H), 0.05 (s, 9H). ¹³C NMR (100 MHz, CDCl₃): δ (ppm) 198.0, 158.8 (br), 154.4, 143.9, 141.7, 137.8, 124.3, 110.9, 81.2, 51.9 (br), 48.2 (br), 28.5, 0.9. IR (CH₂Cl₂, cm⁻¹): 2978, 1701, 1668, 1591, 1414, 1366, 1246, 1162, 1018, 939, 845, 766, 600. HRMS (ESI) calcd for C17H25NO4NaSi [M+Na]+ 358.1451, found 358.1447.

### Tert-butyl 5-oxo-3-(thiophen-3-yl)-4-(trimethylsilyl)-5,6-dihydropyridine-1(2H)-carboxylate (1n)

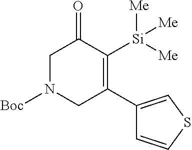 [SMILES: CC(C)(C)OC(=O)N1CC(=O)C([Si](C)(C)C)=C(c2ccsc2)C1]

The general procedure ‘B’ was used with 25.6 mg (0.15 mmol, 0.2 M) of 1-Boc-3-azetidinone, 80.90 mg (0.45 mmol) of trimethyl(thiophen-3-ylethynyl)silane, and 5 mol % of catalyst in toluene. The reaction mixture was purified via flash column chromatography using 15% ethyl acetate in hexanes to afford 42.2 mg of the title compound 1n as slightly pale oil, 80% yield. ¹H NMR (400 MHz, CDCl₃): δ (ppm) 7.37 (m, 1H), 7.28 (s, 1H), 7.09 (d, 1H, J=4 Hz), 4.30 (s, 2H), 4.07 (s, 2H), 1.49 (s, 9H), −0.05 (s, 9H). ¹³C NMR (100 MHz, CDCl₃): δ (ppm) 198.1, 162.7 (br), 154.4, 140.0, 138.1, 127.8, 126.7, 125.6, 81.1, 51.7 (br), 48.7 (br), 28.5, 0.5. IR (CH₂Cl₂, cm⁻¹): 2977, 1701, 1666, 1578, 1411, 1366, 1246, 1163, 1116, 1048, 930, 844, 766, 695. HRMS (ESI) calcd for C17H25NO3NaSSi [M+Na]+ 374.1222, found 374.1227.

### Tert-butyl 5-oxo-4-phenyl-3-(tributylstannyl)-5,6-dihydropyridine-1(2H)-carboxylate (1o)

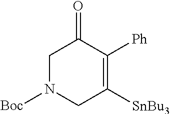

The general procedure ‘B’ was used with 22.9 mg (0.13 mmol, 0.2 M) of 1-Boc-3-azetidinone, 78.48 mg (0.20 mmol) of tributyl(phenylethynyl)stannane, and 5 mol % of catalyst in toluene. The reaction mixture was purified via flash column chromatography using 10-15% ethyl acetate in hexanes to afford 48.5 mg of the title compound 1o as pale oil, 82% yield. ¹H NMR (400 MHz, CDCl₃): δ (ppm) 7.42 (m, 3H), 7.29 (m, 2H), 4.38 (s, 2H), 4.14 (s, 2H), 1.51 (s, 9H), 1.29 (m, 6H), 1.18 (m, 6H), 0.82 (t, 9H, J=7.2 Hz), 0.63 (m, 6H). ¹³C NMR (100 MHz, CDCl₃): δ (ppm) 197.7, 169.0 (br), 154.5, 141.7 (br), 140.4, 129.5, 128.8, 127.7, 81.0, 51.3 (br), 48.5 (br), 29.1, 28.5, 27.4, 13.8, 11.5. IR (CH₂Cl₂, cm⁻¹): 2956, 2924, 2871, 2853, 1703, 1660, 1583, 1415, 1365, 1265, 1239, 1164, 1110, 760, 699. HRMS (ESI) calcd for C28H45NO3NaSn [M+Na]+ 586.2319, found 586.233.

### Tert-butyl 6-benzyl-5-oxo-3,4-dipropyl-5,6-dihydropyridine-1(2H)-carboxylate (4a)

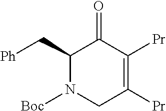

The azetidinone was prepared using a known procedure. Boc-Phe-OH was converted to diazoketone using TMSCHN₂. See, Cesar, J.; Dollenc, M. S. Tet. Lett. 2001, 42, 7099. Notably, yields were not reproducible and were only moderate (30-35%). The diazoketone was converted to the desired azetidinone using Seebach's protocol. See, Podlech, J.; Seebach, D. Helv. Chim. Acta 1995, 78, 1238.

The general procedure ‘A’ was used with 24.0 mg (0.09 mmol, 0.2 M) of (5)-tert-butyl 2-benzyl-3-oxoazetidine-1-carboxylate, 15.18 mg (0.13 mmol) of oct-4-yne, and 5 mol % of catalyst in toluene. The reaction mixture was purified via flash column chromatography using 10% ethyl acetate in hexanes to afford the title compound 4a as pale oil, 88% yield, >99% ee ([α]_{D}²⁰=−27.6 (c=0.44, CHCl₃)). ¹H NMR (400 MHz, CDCl₃): δ (ppm) 7.26 (m, 3H), 7.13 (d, 2H, 8.8 Hz), 4.7 (dd, 1H, 5.6 Hz), 4.60 (d, 1H), 3.70 (d, 1H), 2.85 (m, 2H), 2.25 (m, 4H), 1.52 (sextet, 2H, J=10 Hz), 1.43-1.15 (m, 11H), 0.97 (t, 3H, J=9.6 Hz), 0.93 (t, 3H, J=9.6 Hz). ¹³C NMR (100 MHz, CDCl₃): δ (ppm) 195.4, 155.4, 154.2, 137.3, 133.2, 129.7, 128.7, 126.8, 80.5, 61.7, 43.4, 37.2, 34.4, 28.1, 27.0, 22.7, 21.8, 14.5, 14.4. IR (CH₂Cl₂, cm⁻¹): 3063, 3028, 2963, 2932, 2872, 1697, 1672, 1635, 1495, 1454, 1415, 1368, 1315, 1280, 1243, 1169, 1131, 1030, 976, 949, 878, 762, 700. HRMS (ESI) calcd for C23H33NO3Na [M+Na]+ 394.2358, found 394.2358.

### 1-Methyl-5-phenyl-4-(trimethylsilyl)-1,2,3,6-tetrahydropyridin-3-ol (1l′)

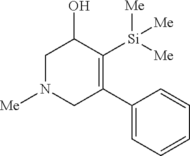 [SMILES: CN1CC(c2ccccc2)=C([Si](C)(C)C)C(O)C1]

The general procedure ‘B’ was used with 33.5 mg (0.19 mmol, 0.2 M) of azetidinone 1, 78.48 mg (0.58 mmol) of alkyne 1, and 5 mol % of catalyst in toluene. After the completion of reaction, solvent was evaporated and the residue was dissolved in ether (0.1M). The resulting solution was cooled to 0° C. and lithium aluminum hydride (10 equiv) was carefully added to it. The resulting suspension was allowed to warm to room temperature, and stirred overnight. The reaction was carefully quenched; the product was extracted with EtOAc and dried over anhydrous MgSO₄. The residue was purified via flash column chromatography using 10% MeOH in DCM to afford the title compound 11′ as colorless oil, 51% yield. ¹H NMR (400 MHz, CDCl₃): δ (ppm) 7.28 (m, 3H), 7.13 (dd, 2H, J=2H), 4.27 (t, 1H, J=4 Hz), 3.18 (d, 1H), 2.9 (d, 1H), 2.80 (dd, 1H, J=3.2, 8.0 Hz), 2.46 (dd, 1H, J=3.2 Hz, 8.0 Hz), 2.36 (s, 3H), 0.15 (s, 9H). ¹³C NMR (100 MHz, CDCl₃): δ (ppm) 150.4, 142.3, 135.1, 128.5, 128.1, 127.6, 67.3, 62.0, 59.9, 45.5, 0.4. IR (CH₂Cl₂, cm⁻¹): 3446, 3053, 2947, 2922, 2843, 2770, 1620, 1594, 1456, 1242, 1114, 1083, 1059, 998, 887, 838, 761, 702. HRMS (ESI) calcd for C15H24NOSi [M+H]+ 262.1627, found 262.1630.

### Tert-butyl 5-hydroxy-3,4-dipropyl-5,6-dihydropyridine-1(2H)-carboxylate (1a′)

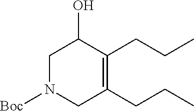 [SMILES: CCCC1=C(CCC)C(O)CN(C(=O)OC(C)(C)C)C1]

The general procedure ‘A’ was used with 51.9 mg (0.30 mmol, 0.2 M) of azetidinone 1, 50.11 mg (0.45 mmol) of alkyne a, and 5 mol % of catalyst in toluene. After the completion of reaction, solvent was evaporated and the residue was dissolved in MeOH (0.1M). To the resulting solution was added CeCl₃.7H₂O (0.55 equiv), then it was cooled to −78° C. and sodium borohydride (1.1 equiv) was carefully added to it. The resulting solution was allowed to warm to room temperature. The reaction was carefully quenched; the product was extracted with EtOAc and dried over anhydrous MgSO₄. The residue was purified via flash column chromatography using 30-50% EtOAc in hexanes to afford the title compound 1a′ as colorless oil, 94% yield. ¹H NMR (400 MHz, CDCl₃): δ (ppm) 3.97 (m, 3H), 3.5 (d, 1H), 3.04 (d, 1H), 2.12 (t, 2H, J=3 Hz), 1.95 (m, 2H), 1.45 (s, 9H), 1.39 (m, 4H), 0.9 (m, 6H). ¹³C NMR (100 MHz, CDCl₃): δ (ppm) 155.8, 132.2, 80.0, 66.2, 48.7 (br), 46.3 (br), 32.6, 31.8, 28.5, 24.1, 22.0, 14.47, 14.41. HRMS (ESI) calcd for C16H29NO3Na [M+Na]+ 306.2045, found 306.2057.

### Example 3

### Crystallographic Characterization of Compound 2c

Crystals of compound 2c, that were suitable for X-ray crystallographic analysis, were grown using THF and hexanes as solvents. A colorless prism shaped crystal 0.35×0.30×0.15 mm in size was mounted on a glass fiber with traces of viscous oil and then transferred to a Nonius KappaCCD diffractometer equipped with Mo Kα radiation (λ=0.71073 Å). Ten frames of data were collected at 150(1)K with an oscillation range of 1 deg/frame and an exposure time of 20 sec/frame (COLLECT Data Collection Software. Nonius B. V. 1998). Indexing and unit cell refinement based on all observed reflection from those ten frames, indicated a monoclinic P lattice. A total of 5667 reflections (Θₘₐₓ=27.52°) were indexed, integrated and corrected for Lorentz, polarization and absorption effects using DENZO-SMN and SCALEPAC (Otwinowski et al. Methods Enzymol. 1997, 276, 307-326). Post refinement of the unit cell gave a=8.7602(13) Å, b=19.148(3) Å, c=10.7909(16) Å, β=110.824(11), and V=1691.8(5) Å³. Axial photographs and systematic absences were consistent with the compound having crystallized in the monoclinic space group P2√a. The structure was solved by a combination of direct methods and heavy atom using SIR 97 ((Release 1.02)—A program for automatic solution and refinement of crystal structure. A. Altomare et al.).

All of the non-hydrogen atoms were refined with anisotropic displacement coefficients. Hydrogen atoms were either located and refined isotropically or assigned isotropic displacement coefficients U(H)=1.2U(C) or 1.5U(Cmethyl), and their coordinates were allowed to ride on their respective carbons using SHELXL97, University of Gottingen, Germany. (Includes SHELXS97, SHELXL97, CIFTAB—Sheldrick, G. M. (1997). Programs for Crystal Structure Analysis (Release 97-2)). The weighting scheme employed was w=1/[σ²(Fₒ²)+(0.0575P)²+1.0249P] where P=(Fₒ²+2F_{c}²)/3. The refinement converged to R1=0.0629, wR2=0.1321, and S=1.1160 for 2305 reflections with 1>2σ(I), and R1=0.1173, wR2=0.1638, and S=1.1160 for 3694 unique reflections and 237 parameters (R1=Σ(∥Fₒ|−|F_{c}∥)/Σ|Fₒ|, wR2=[Σ(w(Fₒ²−F_{c}²)2)/Σ(Fₒ²)²]^{1/2}, and S=Goodness-of-fit on F²=[Σ(w(Fₒ²−F_{c}²)²/(n−p)]^{1/2}, where n is the number of reflections and p is the number of parameters refined). The maximum Δ/σ in the final cycle of the least-squares was 0, and the residual peaks on the final difference-Fourier map ranged from −0.398 to 0.268 e/Å³. Scattering factors were taken from the International Tables for Crystallography, Volume C (Maslen et al. International Tables for Crystallography: Mathematical, Physical and Chemical Tables, Vol. C, Chapter 6, Wilson, A. J. C., Ed.; Kluwer, Dordrecht, The Netherlands, 1992; pp. 476-516; Creagh et al. International Tables for Crystallography: mathematical, Physical and Chemical tables, Vol. C, Chapter 4 Wilson, A. J. C., Ed.; Kluwer, Dordrecht, The Netherlands, 1992; pp. 206-222).

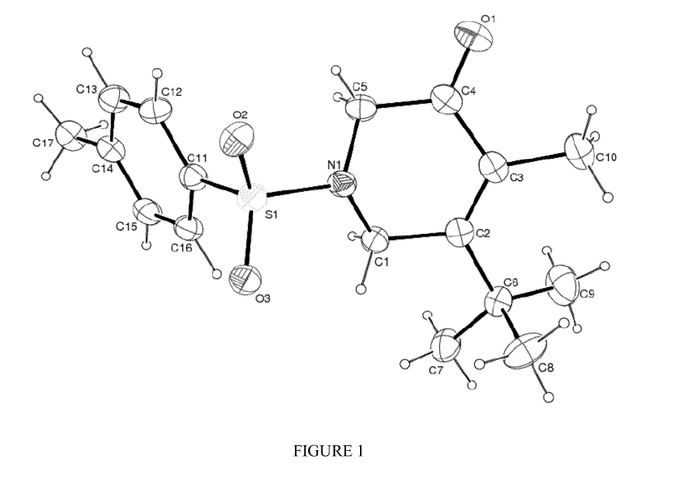

Crystal structure and structural refinement data are shown in Table 2. An ORTEP diagram is illustrated in FIG. 1.

TABLE 2
Crystal data and structure refinement for 2c.

| Empirical formula | C17H23NO3S | |
|---|---|---|
| Formula weight | 321.42 | |
| Temperature | 150(1) K | |
| Wavelength | 0.71073 Å | |
| Crystal system | Monoclinic | |
| Space group | P 2₁/a | |
| Unit cell dimensions | a = 8.7602(13) Å | α = 90°. |
| | b = 19.148(3) Å | β = 110.824(11)°. |
| | c = 10.7909(16) Å | γ = 90°. |

| | |
|---|---|
| Volume | 1691.8(5) Å³ |
| Z | 4 |
| Density (calculated) | 1.262 Mg/m³ |
| Absorption coefficient | 0.203 mm⁻¹ |
| F(000) | 688 |
| Crystal size | 0.35 × 0.30 × 0.15 mm³ |
| Theta range for data collection | 2.28 to 27.52°. |
| Index ranges | −11 <= h <= 11, −20 <= k <= 24, |
| | −14 <= 1 <= 13 |
| Reflections collected | 5667 |
| Independent reflections | 3694 [R(int) = 0.0326] |
| Completeness to theta = 25.00° | 96.9% |
| Absorption correction | Multi-scan |
| Max. and min. transmission | 0.9702 and 0.9323 |
| Refinement method | Full-matrix least-squares on F² |
| Data/restraints/parameters | 3694/0/237 |
| Goodness-of-fit on F² | 1.116 |
| Final R indices [I > 2sigma(I)] | R1 = 0.0629, wR2 = 0.1321 |
| R indices (all data) | R1 = 0.1173, wR2 = 0.1638 |
| Extinction coefficient | 0.042(5) |
| Largest diff. peak and hole | 0.268 and −0.398 e · Å⁻³ |

1. A method of synthesizing a compound of formula (I): 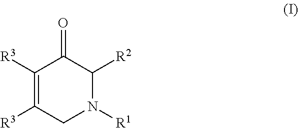
wherein:
R¹ is selected from the group consisting of hydrogen, alkyl or a nitrogen protecting group;
R² is selected from the group consisting of hydrogen, alkyl, alkenyl, alkynyl, cycloalkyl, heterocyclyl, aryl, heteroaryl, silyl and stannyl, any of which may be optionally substituted;
each R³ is independently selected from the group consisting of hydrogen, alkyl, alkenyl, alkynyl, cycloalkyl, heterocyclyl, aryl, heteroaryl, silyl and stannyl, any of which may be optionally substituted, or both R³ are taken together with the atoms to which they are attached to form an optionally substituted ring;
the method comprising combining the following components to form a reaction mixture:
a) a compound of formula (II): 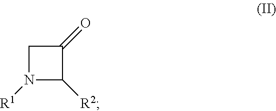
b) a compound of formula (III): 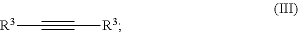
c) a nickel-containing compound; and
d) a ligand.

2. The method of claim 1, wherein the nickel-containing compound comprises nickel(0).

3. The method of claim 2, wherein the nickel-containing compound is bis(cyclooctadiene)nickel(0).

4. The method of claim 1, wherein the ligand is a monophosphine ligand.

5. The method of claim 4, wherein the ligand is triphenylphosphine.

6. The method of claim 1, wherein the reaction mixture further comprises a solvent.

7. The method of claim 6, wherein the solvent is toluene.

8. The method of claim 1, wherein the nickel-containing compound is included in the reaction mixture in an amount of about 1 mol % to about 10 mol %.

9. The method of claim 1, wherein the ligand is included in the reaction mixture in an amount of about 5 mol % to about 20 mol %.

10. The method of claim 1, further comprising heating the reaction mixture.

11. The method of claim 10, wherein the reaction mixture is heated to a temperature of about 25° C. to about 100° C.

12. The method of claim 1, wherein the reaction mixture comprises an inert atmosphere.

13. The method of claim 1, further comprising purifying the compound of formula (I) from the reaction mixture.

14. The method of claim 1, wherein the concentration of the compound of formula (II) in the reaction mixture is about 0.10 M to about 1.0 M.

15. The method of claim 1, wherein R¹ is a nitrogen protecting group.

16. The method of claim 15, wherein R¹ is a tert-butyloxycarbonyl group or a tosyl group.

17. The method of claim 1, wherein the reaction mixture is reacted for about 2 hours to about 10 hours.

18. The method of claim 1, wherein the method provides the compound of formula (I) in about 60% yield to about 99% yield.

19. A compound of formula (I): 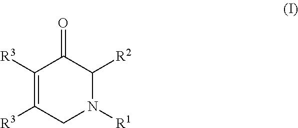
wherein:
R¹ is selected from the group consisting of hydrogen, alkyl or a nitrogen protecting group;
R² is selected from the group consisting of hydrogen, alkyl, alkenyl, alkynyl, cycloalkyl, heterocyclyl, aryl, heteroaryl, silyl and stannyl, any of which may be optionally substituted; and
each R³ is independently selected from the group consisting of hydrogen, alkyl, alkenyl, alkynyl, cycloalkyl, heterocyclyl, aryl, heteroaryl, silyl and stannyl, any of which may be optionally substituted, or both R³ are taken together with the atoms to which they are attached to form an optionally substituted ring.

20. A pharmaceutical composition comprising a compound according to claim 19.
